(19) United States
(12) Patent Application Publication
(10) Pub. No.: US 2022/0380658 A1
(43) Pub. Date: Dec. 1, 2022

(54) FUNCTIONALIZED OLEFIN OLIGOMERS

(71) Applicant: CHEVRON ORONITE COMPANY LLC, San Ramon, CA (US)

(72) Inventors: Curtis B. CAMPBELL, Hercules, CA (US); Andrew R. GIBBS, Novato, CA (US); Andrew M. THOMAS, El Sobrante, CA (US)

(73) Assignee: CHEVRON ORONITE COMPANY LLC, San Ramon, CA (US)

(21) Appl. No.: 17/772,586

(22) PCT Filed: Aug. 31, 2020
(86) PCT No.: PCT/IB2020/058091

§ 371 Date: Apr. 28, 2022

Related U.S. Application Data
(60) Provisional application No. 62/929,026, filed on Oct. 31, 2019.

(51) Int. Cl. C09K 8/584; C10M 145/02; C10M 149/02

(52) U.S. Cl. CPC C09K 8/584; C10M 145/02; C10M 149/02; C10M 2215/086; C10M 2207/262; C09K 2208/34

(57) ABSTRACT

The present disclosure provides a method of treating a hydrocarbon-containing reservoir. The method involves introducing into the reservoir a surfactant composition. The surfactant composition includes an alpha olefin sulfonate or an isomerized olefin sulfonate. The alpha olefin sulfonate or the isomerized olefin sulfonate is synthesized by i) oligomerizing a monomer comprising a C₃ to C₆ mono-olefin to form an oligomerization product and ii) sulfonating the oligomerization product.

### TECHNICAL FIELD

This disclosure relates to functionalized olefin oligomers and commercial applications using compositions that include the same.

### BACKGROUND

Olefin oligomers and their derivatives (e.g., hydrogenated olefin oligomers) are end products or intermediates in the manufacture of a wide variety of commercial products including surfactants, lubricating oils, and additives. The particular olefin oligomer used for a given application typically depends on physical and/or mechanical properties of the olefin oligomer. These properties can be tailored by the specific method used to produce the olefin oligomer and the reaction conditions under which the olefin oligomer was produced.

### SUMMARY

In one aspect, there is provided a method of treating a hydrocarbon-containing reservoir, comprising: introducing into the reservoir a surfactant composition comprising: an alpha olefin sulfonate or an internal olefin sulfonate, wherein the alpha olefin sulfonate or the internal olefin sulfonate is synthesized by i) oligomerizing a monomer comprising a C₃ to C₆ mono-olefin to form an oligomerization product and ii) sulfonating the oligomerization product.

In another aspect, there is provided a surfactant for enhanced oil recovery, the surfactant comprising: an alpha olefin sulfonate or an internal olefin sulfonate, wherein the alpha olefin sulfonate or the internal olefin sulfonate is synthesized by i) oligomerizing a monomer comprising a C₃ to C₆ mono-olefin to form an oligomerization product and ii) sulfonating the oligomerization product.

In yet another aspect, there is provided a lubricating oil composition comprising: a base oil; and a succinimide dispersant synthesized by i) oligomerizing a monomer comprising a C₃ to C₆ mono-olefin to form an oligomerization product and ii) functionalizing the oligomerization product with an ethylenically saturated carboxylic acid group.

In still yet another aspect, there is provided a dispersant composition comprising: a succinimide dispersant synthesized by i) oligomerizing a monomer comprising a C₃ to C₆ mono-olefin to form an oligomerization product and ii) functionalizing the oligomerization product with an ethylenically saturated carboxylic acid group.

In still yet another further aspect, there is provided a lubricating oil composition comprising: a base oil; and a detergent additive synthesized by i) oligomerizing a monomer comprising a C₃ to C₆ mono-olefin to form an oligomerization product and ii) alkylating a hydroxyaromatic compound with the oligomerization product.

In still yet another further additional aspect, there is provided a detergent composition comprising: a detergent additive synthesized by i) oligomerizing a monomer comprising a C₃ to C₆ mono-olefin to form a oligomerization product and ii) alkylating a hydroxyaromatic compound with the oligomerization product.

In still yet another further additional other aspect, there is provided a surfactant composition comprising: an alcohol ether sulfate or an alcohol ether carboxylate synthesized by i) oligomerizing a monomer comprising a C₃ to C₆ mono-olefin to form a oligomerization product and ii) converting the oligomerization product to an alcohol.

### BRIEF DESCRIPTION OF DRAWINGS

FIG. 1 shows the Electrospray Ionization (ESI) mass spectrum of the sodium sulfonate composition prepared in Example 6.

### DETAILED DESCRIPTION

The present invention provides functionalized olefin oligomers and uses thereof in a variety of commercial applications. At least some of the functionalized olefin oligomers are important because of their ability to be used as surfactants for chemical enhanced oil recovery (CEOR). CEOR typically employees anionic surfactants, which include, but are not limited to, alkyl aromatic sulfonates (AAS), alpha olefin sulfonates (AOS), internal olefin sulfonates (IOS), alcohol sulfates, alkyl or alcohol ether sulfates (AES), and alkyl or alcohol ether carboxylates (AEC). Some olefin oligomers can also be used as oil additives, lubricants, anti-fogging or wetting additives, and adhesion promoters. Olefin oligomers can also be used as plasticizers, soaps, detergents, fabric softeners, anti-statics as well as many other uses.

Monomers

The functionalized olefin oligomers can be produced by oligomerizing olefin monomers to form olefin oligomer and functionalizing product of the oligomerization.

A wide range of monomers comprising, consisting essentially of, or consisting of, C₃ to C₆ mono-olefins can be oligomerized. Suitable monomers include internal olefins, alpha olefins, trisubstituted olefins, any mixtures thereof and the like. Further, the alpha olefins can comprise, or consist essentially of, normal alpha olefins (sometimes referred to as “linear alpha olefins”).

Generally, the monomer can comprise (or consist essentially of, or consist of) C₃ to C₆ mono-olefins, C₃ to C₅ mono-olefins, or C₃ to C₄ mono-olefins. In other embodiments, the monomer can comprise (or consist essentially of, or consist of) C₃ olefins; alternatively, C₄ mono-olefins, alternatively, C₅ mono-olefins, or alternatively, C₆ mono-olefins. Thus, mixtures of olefins having different numbers of carbon atoms can be used, or olefins having predominantly a single number of carbon atoms can be used as the monomer.

The monomer can comprise at least 50 wt. %, at least 55 wt. %, at least 60 wt. %, at least 65 wt. %, at least 70 wt. %, at least 75 wt. %, at least 80 wt. %, at least 85 wt. %, at least 90 wt. %, or at least 95 wt. % of any olefin or combination of olefins described herein. Additionally or alternatively, the monomer can comprise a maximum of 100 wt. %, 99 wt. %, 98 wt. %, 97 wt. %, or 96 wt. %, of any olefin, or combination of olefins described herein. Generally, the weight percent can be in a range from any minimum weight percent disclosed herein to any maximum weight percent disclosed herein. Therefore, non-limiting monomer weight percent ranges can include the following ranges: from 50 to 100 wt. %, from 55 to 99 wt. %, from 60 to 98 wt. %, from 65 to 97 wt. %, from 70 to 96 wt. %, from 75 to 100 wt. %, from 80 to 100 wt. %, or from 80 to 98 wt. % of any olefin, or mixture of olefins described herein.

The olefins can be cyclic or acyclic, and/or linear or branched. For example, the monomer can comprise, consist essentially of, or consist of, acyclic olefins; additionally or alternatively, the monomer can comprise, consist essentially of, or consist of, linear olefins.

The monomer can comprise (or consist essentially of, or consist of) propylene, 1-butene, isobutylene, 1-pentene, 2-methyl-1-butene, 2-methyl-2-butene or any combination thereof; alternatively, propylene; alternatively, 1-butene; or alternatively, isobutylene. Thus, mixtures of olefins having different numbers of carbon atoms can be used, or olefins having predominantly a single number of carbon atoms can be used as the monomer.

The oligomerization reaction generally involves introducing a monomer comprising a C₃ to C₆ mono-olefin and a catalyst into a reaction zone, and oligomerizing the monomer to form an olefin oligomer in the reaction zone. In general, any suitable catalyst can be used. Suitable catalysts include, but are not limited to, ionic liquid catalysts, phosphoric acid, zeolite, mesoporous aluminosilicate, or Ziegler-Natta catalysts. There can be more than catalyst used. A more detailed discussion of ionic liquid catalysts can be found in U.S. Pat. No. 9,938,473, which is hereby incorporated by reference. A more detailed description of phosphoric acid catalysts can be found in U.S. Pat. Nos. 2,592,428, 2,814,655, 3,887,634, and 8,183,192, which are hereby incorporated by reference. A more detailed discussion of zeolite catalysts can be found in U.S. Pat. No. 4,547,612, which is incorporated here by reference. A more detailed discussion of mesoporous aluminosilicate catalysts can be found in WO 201120968, which is incorporated here by reference. A more detailed discussion of Ziegler-Natta catalysts can be found in EP 638593, and is incorporated here by reference. Various methods to oligomerize olefins are described in Skupinska, J. Chem. Rev. 1991, 91, 613-648, and is incorporated here by reference.

Olefin Oligomer Product

The olefin oligomer can comprise dimers, trimers, and/or higher oligomers. In some embodiments, the olefin oligomer can comprise (i) at least 75 wt %, 80 wt %, 85 wt %, 90 wt %, or 95 wt % dimers, trimers, tetramers, pentamers, hexamers, heptamers, octamers, nonamers, and/or decamers; (ii) at least 50 wt %, 55 wt %, 60 wt %, 65 wt %, 70 wt %, 80 wt %, 85 wt %, or 90 wt % trimers, tetramers, pentamers, hexamers, heptamers, octamers, nonamers, and/or decamers; (iii) at least 75 wt %, 80 wt %, 85 wt %, 90 wt %, or 95 wt % dimers, trimers, tetramers, pentamers, hexamers, and/or heptamers; (iv) at least 50 wt %, 55 wt %, 60 wt %, 65 wt %, 70 wt %, 80 wt %, 85 wt %, or 90 wt % trimers, tetramers, pentamers, hexamers, and/or heptamers; (v) at least 30 wt %, 35 wt %, 40 wt %, 45 wt %, 50 wt %, 55 wt %, or 60 wt % dimers, trimers, tetramers, and/or pentamers; (vi) at least 25 wt %, 30 wt %, 35 wt %, 40 wt %, 45 wt %, or 50 wt % trimers, tetramers, and/or pentamers; (vii) at least 25 wt %, 30 wt %, 35 wt %, 40 wt %, 45 wt %, 50 wt %, 55 wt %, or 60 wt % dimers, trimers, and/or tetramers; (viii) at least 20 wt %, 25 wt %, 30 wt %, 35 wt %, 40 wt %, 45 wt %, or 50 wt % trimers and/or tetramers; or (ix) any combination thereof.

In additional or alternative embodiments, the olefin oligomer can comprise a total of at least 35 wt %, 45 wt %, 45 wt %, 50 wt %, 55 wt %, 60 wt %, or 65 wt % trimer, tetramer and pentamer; alternatively or additionally, a maximum total of 100 wt %, 95 wt %, 90 wt %, or 85 wt % trimer, tetramer and pentamer. In some embodiments, the olefin oligomer can comprise a total of from 35 wt % to 100 wt %, from 40 wt % to 95 wt %, from 45 wt % to 90 wt %, from 40 wt % to 85 wt %, from 50 wt % to 90 wt %, or from 50 wt % to 85 wt %, trimer, tetramer and pentamer.

The olefin oligomer can comprise less than 40 wt %, 30 wt %, 25 wt %, 20 wt %, 18 wt %, 16 wt %, 14 wt %, 12 wt %, or 10 wt % dimer. Additionally or alternatively, the olefin oligomer can comprise less than 30 wt %, 25 wt %, 20 wt %, 15 wt %, 10 wt %, 8 wt %, 6 wt %, 5 wt %, 4 wt %, 3 wt %, or 2 wt % oligomer containing 7 or more monomer units.

In some aspects, the olefin oligomer can comprise at least 50 wt %, 60 wt %, 70 wt %, 75 wt %, 80 wt %, 85 wt %, 90 wt %, or 95 wt % C₁₂ to C₇₀ (e.g., C₁₂ to C₄₀, C₁₂ to C₃₀, C₁₂ to C₂₀, C₁₄ to C₇₀, C₁₄ to C₄₀, C₁₄ to C₃₀, C₁₄ to C₂₀, C₁₆ to C₇₀, C₁₆ to C₄₀, C₁₆ to C₃₀, C₁₆ to C₂₄, C₂₀ to C₇₀, C₂₀ to C₄₀, C₂₀ to C₃₀, or C₂₀ to C₂₄) oligomers. In some aspects, the olefin oligomer can comprise less than 30 wt %, 25 wt %, 20 wt %, 15 wt %, 10 wt %, 8 wt %, 6 wt %, 5 wt. %, 4 wt %, 3 wt %, or 2 wt %>C₇₀ oligomers. The wt % of the oligomer(s) disclosed herein is based upon the total weight of the olefin oligomer.

The olefin oligomer can be a propylene oligomer (i.e., the repeating units of the olefin oligomer can be substantially all propylene units). For example, the repeating units of the oligomer can contain at least about 90 mol %, at least 95 mol %, at least 98 mol %, or at least 99 mol % propylene units.

The olefin oligomer can be an isobutylene oligomer (i.e., the repeating units of the olefin oligomer can be substantially all isobutylene units). For example, the repeating units of the oligomer can contain at least 75 mol %, at least 90 mol %, at least 95 mol %, at least 98 mol %, or at least 99 mol % isobutylene units.

The olefin oligomer can have a number average molecular weight (Mₙ) in a range from 150 to 10,000 g/mol. For instance, the Mₙ of the olefin oligomer or can be at least 150, 250, 325, 400, 500, 600, 650, 700, or 750 g/mol. Additionally or alternatively, the maximum Mₙ can be 10,000, 7500, 6000, 5000, 4000, 3000, 2500, or 2000 g/mol. Generally, the Mₙ of the olefin oligomer can be in a range from any minimum Mₙ disclosed herein to any maximum Mₙ disclosed herein.

The olefin oligomer can have a viscosity index (ASTM D2270) of at least 80. For instance, the viscosity index of the olefin oligomer can be at least 85, 90, 95, 100, or 110. Additionally or alternatively, the maximum viscosity index can be 200, 175, 150, 140, 135, 130, 125, or 120. Generally, the viscosity index of the olefin oligomer can be in a range from any minimum viscosity index disclosed herein to any maximum viscosity index disclosed herein.

The olefin oligomer can have any suitable kinematic viscosity at 100° C. (ASTM D445), for instance, ranging from 1.5 to 50 mm²/s. For instance, the olefin oligomer or the hydrogenated olefin oligomer can have a kinematic viscosity at 100° C. of at least 2, 3, 4, or 6 mm²/s. Additionally or alternatively, the maximum kinematic viscosity at 100° C. of the oligomer can be 50, 20, 14, 12, 10, or 8 mm²/s. Generally, the kinematic viscosity at 100° C. of the olefin oligomer can be in a range from any minimum kinematic viscosity disclosed herein to any maximum kinematic viscosity disclosed herein.

The pour point (ASTM D97) of the olefin oligomer can be within a range from −5° C. to −60° C. For instance, the minimum pour point of the olefin oligomer can be −60° C., −50° C., −45° C., −40° C., or −35° C. Additionally or alternatively, the maximum pour point can be −5° C., −8° C., −10° C., −15° C., or −20° C. Generally, the pour point of the olefin oligomer can be in a range from any minimum pour point temperature disclosed herein to any maximum pour point temperature disclosed herein.

### Functionalized Olefin Oligomer

Heteroatom-Functionalized Oligomers

The olefin oligomer may be functionalized by reacting a heteroatom-containing group with the olefin oligomer with or without a catalyst. These reactions include hydroxylation, hydrosilation, ozonolysis, hydroformylation, hydroamidation, sulfonation, halogenation, hydrohalogenation, hydroboration, epoxidation, Diels-Alder reactions with polar dienes, Friedel-Crafts reactions with polar aromatics (e.g., hydroxyaromatics), and maleation with activators such as free radical generators (e.g., peroxides).

Exemplary heteroatom-containing groups include alcohols, amines, aldehydes, hydroxyaromatic compounds, sulfonates, acids and anhydrides.

The number of functional groups in the resulting heteroatom-functionalized oligomer can be in a range of 0.60 to 1.2 functional groups per chain (e.g., 0.75 to 1.1 functional groups per chain). The number of functional groups per chain can be determined by any conventional method (e.g., ¹H NMR spectroscopy).

Detergent Alcohols

The olefin oligomers can be functionalized to prepare detergent alcohols such as alkyl (or alcohol) ether sulfates (AES), alkyl (or alcohol) ether carboxylates (AEC), and alkyl sulfates (AS). Detergent alcohols and their derivatives are widely used as raw materials in the production of surfactants for laundry and dishwashing detergents, and other household cleaners and shampoos. These oligomers are also widely used in the cosmetics and toiletries industries.

Alcohols can be prepared from the olefin oligomer which can be used as feedstocks for preparing high molecular weight polyethers. The polyethers can be subsequently converted to AES and AEC. These compositions can be used as surfactants for chemical EOR applications. Detailed description of these compounds can be found in C. Negin et al. (Petroleum 2017, 3, 197-211) and U.S. Pat. No. 9,745,259, the relevant portions of which are hereby incorporated by reference.

In a conventional process, the olefin oligomer can be converted to primary alcohols via oxo chemistry. For example, by reaction with ethylene oxide, the alcohols can form a variety of nonionic ethoxylates, which may themselves serve as surfactants or be further derivatized. Alcohol ether sulfates can be derived from the sulfation of the ethoxylates. Alternatively, the alcohols may be directly sulfated to produce alkyl sulfates (AS).

Olefin Sulfonates

Olefin sulfonate surfactants (e.g., alpha olefin sulfonate and internal olefin sulfonate) provide have favorable detergency, high compatibility with hard water, and good wetting and foaming properties. Commercial applications include shampoos, light-duty liquid detergents, bubble baths, and heavy-duty liquid, powder detergents, and emulsion polymerization. In particular, C₁₄-C₁₆ alpha olefin sulfonate (AOS) blends are frequently used in liquid hand soaps. Due to their good detergency, foaming and wetting properties, olefin sulfonates can be utilized as surfactants in chemical EOR applications and in household detergents.

Olefin oligomers can be precursors in the production of AOS surfactants. The olefin oligomer may be functionalized by reaction of the oligomer with a sulfonation reagent to provide an olefin sulfonic acid intermediate which can then be neutralized to provide an olefin sulfonate.

For internal olefin sulfonates (IOS), the sulfonation reaction can take place at any place along the chain since its double bond is randomly distributed. IOS can be produced by the sulfonation of internal olefins.

Sulfonation of the olefin oligomer may be performed by any known method. For example, olefin oligomers can be first sulfonated in a continuous thin film reactor to produce a mixture of alkene sulfonic acids and sultones (cyclic sulfonate esters).

Sulfonation can also occur by using chlorosulfonic acid, sulfamic acid, and sulfuric acid/oleum.

Neutralization of the olefin sulfonic acid may be carried out in a continuous or batch process by any method known to one skilled in the art to produce the olefin sulfonate. Typically, an olefin sulfonic acid is neutralized with a source of a mono-covalent cation (e.g., an alkali metal such as sodium or ammonium or substituted ammonium ion) then hydrolyzed at elevated temperatures to convert the remaining sultones to alkene sulfonates and hydroxy sulfonates. This results in an aqueous solution of olefin sulfonates. If a solid anhydrous product is desired, it can be obtained by neutralizing and hydrolyzing the solution in isopropanol instead of water. Optionally, the neutralized olefin sulfonate may be further hydrolyzed with additional base or caustic.

The surfactant composition may also comprise of an aqueous base such as carbonates, hydroxides, bicarbonates of alkali metal ion, ammonium ion, and amine compounds.

Depending on the type of reservoir, alkali may be included with the surfactant composition. In one embodiment, the alkali employed is a basic salt of an alkali metal from Group IA metals of the Periodic Table, such as an alkali metal hydroxide, borate, carbonate or bicarbonate. For example, the alkali may include sodium carbonate, sodium bicarbonate, sodium silicate, tetrasodium EDTA, sodium metaborate, sodium citrate, or sodium tetraborate. Use of the alkali maintains the surfactant in a high pH environment, which prolong the stability of the surfactant or can minimize surfactant adsorption. Alkali can also protect the surfactant from hardness.

The surfactant composition may also include additional additives, such as co-surfactants, polymers, chelators, co-solvents, reducing agents/oxygen scavengers, and biocides. This combined composition is often referred to as a slug.

Suitable co-solvents may be selected from lower carbon chain alcohols like isopropyl alcohol, ethanol, n-propyl alcohol, n-butyl alcohol, sec-butyl alcohol, n-amyl alcohol, sec-amyl alcohol, n-hexyl alcohol, sec-hexyl alcohol and the like: alcohol ethers, polyalkylene alcohol ethers, poly alkylene glycols, poly(oxyalkylene)glycols, poly(oxyalkylene)glycols ethers or any other common organic co-solvent or combinations of any two or more co-solvents. In some instances, the cosolvent may be water.

In particular, polymers may be used to control the mobility of the slug when injected into a reservoir for enhanced oil recovery. Suitable polymers include, but are not limited to, biopolymers such as xanthan gum and scleroglucan and synthetic polymers such as water soluble unhydrolyzed or partially hydrolyzed polyacrylamides (HPAMs or PHPAs) and hydrophobically modified associated polymers. Also included are co-polymers of polyacrylamide (PAM) and one or both of 2-acrylamido-2-methylpropane sulfonic acid (and/or sodium salt) sold under trademark AMPS (also more generally known as acrylamido tertiobutyl sulfonic acid or ATBS) and N-vinyl pyrrolidone (NVP).

Chelators may be added to complex with multivalent cations and soften the water in the surfactant composition. Examples of chelators include ethylenediaminetetraacetic acid (EDTA) which can also be used as an alkali, methylglycinediacetic acid (MGDA). Chelants may be utilized to handle hard brines. The amount of chelant may be selected based on the amount of divalentions in the surfactant solutions.

Reducing agents/oxygen scavengers such as sodium dithionite may be added to remove any oxygen in the mixture and reduce any free iron into Fe²⁺. They can be used to protect synthetic polymers from reactions that cleave the polymer molecule and lower or remove viscosifying abilities. A reduced environment can also lower surfactant adsorption.

Biocides can be added to prevent organic (algal) growth in facilities, stop sulfate reducing bacteria (SRB) growth which “sour” the reservoir by producing dangerous and deadly H₂S, and are also used to protect biopolymers from biological life which feed on their sugar-like structures and therefore remove mobility control. Biocides include aldehydes and quaternary ammonium compounds.

Alkyl-Substituted Hydroxyaromatic Compounds

The olefin oligomer described herein may be functionalized by alkylation of a hydroxyaromatic compound with the olefin oligomer to form an alkyl-substituted hydroxyaromatic compound. Alkyl-substituted hydroxyaromatic compounds and their salts are useful as lubricant additives.

The alkyl-substituted hydroxyaromatic compound is prepared by alkylation methods that are well known in the art. Useful hydroxyaromatic compounds that may be alkylated include mononuclear monohydroxy and polyhydroxy aromatic hydrocarbons having 1 to 4, and preferably 1 to 3, hydroxyl groups. Suitable hydroxyaromatic compounds include phenol, catechol, resorcinol, hydroquinone, pyrogallol, cresol, and the like and mixtures thereof.

Alkylation of the hydroxyaromatic compound with the olefin oligomer is generally carried out in the presence of an alkylation catalyst. Useful alkylation catalysts include Lewis acids, solid acids, trifluoromethanesulfonic acid, and acidic molecular sieve catalysts. Suitable Lewis acids include aluminum trichloride, boron trifluoride and boron trifluoride complexes (e.g., boron trifluoride etherate, boron trifluoride-phenol and boron trifluoride-phosphoric acid. Suitable solid acids include the sulfonated acidic ion exchange resin type catalysts such as AMBERLYSTO®-36 (Dow Chemical Company), clay catalysts (e.g. CelaClear F-24X Engineered Clays Corp) or zeolite materials.

The reaction conditions for the alleviation depend upon the type of catalyst used, and any suitable set of reaction conditions that result in high conversion to the alkyl hydroxyaromatic product can be employed. Typically, the reaction temperature for the alkylation reaction will be in the range of from 15° C. to 200° C. (e.g., 85° C. to 135° C.). The reaction pressure will generally be atmospheric, although higher or lower pressures may be employed. The alkylation process can be practiced in a batch wise, continuous or semi-continuous manner. The molar ratio of the hydroxyaromatic compound to the olefin oligomer may be in the range of 10:1 to 0.5:1 (e.g., 5:1 to 3:1).

The alkylation reaction may be carried out neat or in the presence of a solvent which is inert to the reaction of the hydroxyaromatic compound and the olefin mixture.

Upon completion of the reaction, the desired alkyl-substituted hydroxyaromatic compound can be isolated using conventional techniques.

The alkyl group of the alkyl-substituted hydroxyaromatic compound is typically attached to the hydroxyaromatic compound primarily in the ortho and para positions, relative to the hydroxyl group. The alkyl-substituted hydroxyaromatic compound may contain 1 to 99% ortho isomer and 99 to 1% para isomer (e.g., 5 to 70% ortho isomer and 95 to 30% para isomer).

Metal salts of alkylphenols (i.e., phenates) are a useful class of detergent. These detergents can be made by reacting an alkaline earth metal hydroxide or oxide (e.g., CaO, Ca(OH)₂, BaO, Ba(OH)₂, MgO, Mg(OH)₂) with an alkylphenol or sulfurized alkylphenol. When a non-sulfurized alkylphenol is used, the sulfurized product may be obtained by methods well known in the art. These methods include heating a mixture of alkylphenol and sulfurizing agent (e.g., elemental sulfur, sulfur halides such as sulfur dichloride, and the like) and then reacting the sulfurized alkylphenol with an alkaline earth metal base.

Metal salts of alkyl-substituted hydroxyaromatic carboxylic acids are also useful as detergents. Alkyl-substituted hydroxyaromatic carboxylic acids are typically prepared by carboxylation, for example by the Kolbe-Schmitt process, of alkyl-substituted phenoxides.

Non-limiting examples of suitable metals include alkali metals, alkaline earth metals and transition metals. Examples include Li, Na, K, Mg, Ca, Zn, Co, Mn, Zr, Ba, and B.

Many detergent compositions are overbased, containing large amounts of a metal base that is achieved by reacting an excess of a metal compound (e.g., a metal carbonate, hydroxide or oxide) with an acidic gas (e.g., carbon dioxide). Useful detergents can be neutral, mildly overbased, or highly overbased. Processes for overbasing are known to those skilled in the art.

The basicity of the detergents may be expressed as a total base number (TBN). A total base number is the amount of acid needed to neutralize all of the basicity of the overbased material. The TBN may be measured using ASTM D2896 or an equivalent procedure. The detergent may have a low TBN (i.e. a TBN of less than 50 mg KOH/g), a medium TBN (i.e. a TBN of 50 to 150 mg KOH/g) or a high TBN (i.e. a TBN of greater than 150 mg KOH/g, such as 150 to 500 mg KOH/g or more).

Olefin Oligomer Grafted with an Ethylenically Unsaturated Carboxylic Acid Material

The olefin oligomer described herein may be functionalized by reaction of the oligomer with an ethylenically unsaturated carboxylic acid or a derivative thereof.

The ethylenically unsaturated carboxylic acid or a derivative thereof may be an acid or anhydride or derivatives thereof that may be wholly esterified, partially esterified or mixtures thereof. When partially esterified, other functional groups include acids, salts or mixtures thereof. Suitable salts include alkali metals, alkaline earth metals or mixtures thereof.

Suitable examples of the ethylenically unsaturated carboxylic acid or derivatives thereof include (meth)acrylic acid, methyl acrylate, maleic acid or anhydride, fumaric acid, itaconic acid or anhydride or mixtures thereof, or substituted equivalents thereof.

Functionalization of the olefin oligomer with the ethylenically unsaturated carboxylic acid or derivatives thereof can be achieved by any suitable method. For example, the ethylenically unsaturated carboxylic acid or derivatives thereof may be grafted onto the olefin oligomer by a process involving the use of chlorine or by a thermal “ene” process or a free radical process.

Upon reaction with the oligomer, the double bond of the ethylenically unsaturated carboxylic acid or derivatives thereof becomes saturated. Thus, for example, maleic anhydride reacted with the olefin oligomer becomes an alkyl-substituted succinic anhydride.

The alkyl-substituted succinic anhydride can then be used as feedstock to make succinimide dispersants. Succinimide dispersants keep vital engine parts clean, prolonging engine life and helping to maintain proper emissions and good fuel economy. Succinimides as additives can also provide protection against abrasive, soot promoted engine wear in diesel engine oil formulations. It can also provide excellent soot dispersancy and act as an oil viscosity index improver.

The functionalized olefin oligomer can in turn be derivatized with a derivatizing compound. The derivatizing compound can react with functional groups of the functionalized oligomer by means such as nucleophilic substitution, Mannich base condensation, and the like. Exemplary derivatizing compounds include amines, hydroxyl-containing compounds, metal salts, anhydride-containing compounds and acetyl halide-containing compounds. The derivatizing compound can contain one or more nucleophilic groups. A derivatized oligomer can be made by contacting a functionalized oligomer (i.e., substituted with a carboxylic acid/anhydride or ester) with a nucleophile (i.e., amine, alcohol, including polyols, aminoalcohols, reactive metal compounds and the like).

Amine compounds useful as nucleophiles for reaction with the functionalized oligomer include mono- and polyamines having about 2 to 60 (e.g., 3 to 20) total carbon atoms and about 1 to 12 (e.g., 3 to 9) nitrogen atoms. Suitable polyamines include aliphatic polyamines, cycloaliphatic polyamines, aromatic polyamines, ether group-containing aliphatic polyamines, and polyoxyalkylene polyamines, for example, available under the name JEFFAMINE® (from Huntsman International LLC, USA).

Exemplary polyamines are those having the formula: H₂N—(R′NH)—H wherein R′ is a straight- or branched-chain alkylene group having 2 or 3 carbon atoms and x is 1 to 9 (e.g., ethylenediamine, diethylenetriamine, triethylenetetraamine, tetraethylenepentamine, pentaethylene hexamine, and heavy polyamines such as heavy polyamine X, available from Dow Chemical Company).

The functionalized oligomers and/or derivatized oligomer have uses as lubricating oil additives which can act as dispersants, viscosity index improvers, or multifunctional viscosity index improvers.

Functionalized oligomers and/or derivatized oligomers having uses as dispersants typically have a number average molecular weight (Mₙ) of 10,000 g/mol or less and can typically range from 500 to 10,000 g/mol, 750 to 5000 g/mol, or 1000 to 3000 g/mol).

The functionalized oligomers and/or derivatized oligomers described herein may be combined with other additives (e.g., detergents, dispersants, oxidation inhibitors, wear inhibitors, friction modifiers, rust inhibitors, viscosity modifiers, pour point depressants, foam inhibitors, and the like to form compositions for many applications, including lubricating oil additive packages, lubricating oils, and the like.

Compositions containing these additives are typically blended into a base oil in amounts which are effective to provide their normal attendant function. Typical amounts of such additives are shown in Table 1 below. The weight amounts in the table below, as well as other amounts mentioned herein, are directed to the amount of active ingredient (that is the non-diluent portion of the ingredient). The weight percent (wt. %) indicated below is based on the total weight of the lubricating oil composition.

Lubricating Oil

The olefin oligomers of the present disclosure may be useful as additives (e.g., as dispersants, detergents, etc.) in lubricating oils to prevent or reduce undesirable ignition events in combustion engines. When employed in this manner, the additives are usually present in the lubricating oil composition in concentrations ranging from 0.001 to 10 wt. % (including, but not limited to, 0.01 to 5 wt. %, 02 to 4 wt. %, 0.5 to 3 wt. %, 1 to 2 wt. %, and so forth), based on the total weight of the lubricating oil composition. If other hydride donors are present in the lubricating oil composition, a lesser amount of the additive may be used.

Oils used as the base oil will be selected or blended depending on the desired end use and the additives in the finished oil to give the desired grade of engine oil, e.g. a lubricating oil composition having an Society of Automotive Engineers (SAE) Viscosity Grade of 0 W, 0 W-8, 0 W-16, 0 W-20, 0 W-30, 0 W-40, 0 W-50, 0 W-60, 5 W, 5 W-20, 5 W-30, 5 W-40, 5 W-50, 5 W-60, 10 W, 10 W-20, 10 W-30, 10 W-40, 10 W-50, 15 W, 15 W-20, 15 W-30, or 15 W-40.

The oil of lubricating viscosity (sometimes referred to as “base stock” or “base oil”) is the primary liquid constituent of a lubricant, into which additives and possibly other oils are blended, for example to produce a final lubricant (or lubricant composition). A base oil, which is useful for making concentrates as well as for making lubricating oil compositions therefrom, may be selected from natural (vegetable, animal or mineral) and synthetic lubricating oils and mixtures thereof.

Definitions for the base stocks and base oils in this disclosure are the same as those found in American Petroleum Institute (API) Publication 1509 Annex E (“API Base Oil Interchangeability Guidelines for Passenger Car Motor Oils and Diesel Engine Oils,” December 2016). Group I base stocks contain less than 90% saturates and/or greater than 0.03% sulfur and have a viscosity index greater than or equal to 80 and less than 120 using the test methods specified in Table E-1. Group II base stocks contain greater than or equal to 90% saturates and less than or equal to 0.03% sulfur and have a viscosity index greater than or equal to 80 and less than 120 using the test methods specified in Table E-1. Group III base stocks contain greater than or equal to 90% saturates and less than or equal to 0.03% sulfur and have a viscosity index greater than or equal to 120 using the test methods specified in Table E-1. Group IV base stocks are polyalphaolefins (PAO). Group V base stocks include all other base stocks not included in Group I, II, III, or IV.

Natural oils include animal oils, vegetable oils (e.g., castor oil and lard oil), and mineral oils. Animal and vegetable oils possessing favorable thermal oxidative stability can be used. Of the natural oils, mineral oils are preferred. Mineral oils vary widely as to their crude source, for example, as to whether they are paraffinic, naphthenic, or mixed paraffinic-naphthenic. Oils derived from coal or shale are also useful. Natural oils vary also as to the method used for their production and purification, for example, their distillation range and whether they are straight run or cracked, hydrorefined, or solvent extracted.

Synthetic oils include hydrocarbon oil. Hydrocarbon oils include oils such as polymerized and interpolymerized olefins (e.g., polybutylenes, polypropylenes, propylene isobutylene copolymers, ethylene-olefin copolymers, and ethylene-alphaolefin copolymers). Polyalphaolefin (PAO) oil base stocks are commonly used synthetic hydrocarbon oil. By way of example, PAOs derived from C₈ to C₁₄ olefins, e.g., C₈, C₁₀, C₁₂, C₁₄ olefins or mixtures thereof, may be utilized.

Other useful fluids for use as base oils include non-conventional or unconventional base stocks that have been processed, preferably catalytically, or synthesized to provide high performance characteristics.

Non-conventional or unconventional base stocks/base oils include one or more of a mixture of base stock(s) derived from one or more Gas-to-Liquids (GTL) materials, as well as isomerate/isodewaxate base stock(s) derived from natural wax or waxy feeds, mineral and or non-mineral oil waxy feed stocks such as slack waxes, natural waxes, and waxy stocks such as gas oils, waxy fuels hydrocracker bottoms, waxy raffinate, hydrocrackate, thermal crackates, or other mineral, mineral oil, or even non-petroleum oil derived waxy materials such as waxy materials received from coal liquefaction or shale oil, and mixtures of such base stocks.

Base oils for use in the lubricating oil compositions of present disclosure are any of the variety of oils corresponding to API Group I, Group II, Group III, Group IV, and Group V oils, and mixtures thereof, preferably API Group II, Group IIII, Group IV, and Group V oils, and mixtures thereof, more preferably the Group III to Group V base oils due to their exceptional volatility, stability, viscometric and cleanliness features.

Typically, the base oil will have a kinematic viscosity at 100° C. (ASTM D445) in a range of 2.5 to 20 mm²/s (e.g., 3 to 12 mm²/s, 4 to 10 mm²/s, or 4.5 to 8 mm²/s).

The present lubricating oil compositions may also contain conventional lubricant additives for imparting auxiliary functions to give a finished lubricating oil composition in which these additives are dispersed or dissolved. For example, the lubricating oil compositions can be blended with antioxidants, ashless dispersants, anti-wear agents, detergents such as metal detergents, rust inhibitors, dehazing agents, demulsifying agents, friction modifiers, metal deactivating agents, pour point depressants, viscosity modifiers, antifoaming agents, co-solvents, package compatibilizers, corrosion-inhibitors, dyes, extreme pressure agents and the like and mixtures thereof. A variety of the additives are known and commercially available. These additives, or their analogous compounds, can be employed for the preparation of the lubricating oil compositions of the invention by the usual blending procedures.

Each of the foregoing additives, when used, is used at a functionally effective amount to impart the desired properties to the lubricant. Thus, for example, if an additive is an ashless dispersant, a functionally effective amount of this ashless dispersant would be an amount sufficient to impart the desired dispersancy characteristics to the lubricant. Generally, the concentration of each of these additives, when used, may range, unless otherwise specified, from about 0.001 to about 20 wt. %, such as about 0.01 to about 10 wt. %.

TABLE 1

| Compound | Typical, wt % | Preferred, wt. % |
|---|---|---|
| Detergent | 0.1 to 20 | 0.1 to 8 |
| Dispersant | 0.1 to 20 | 0.1 to 8 |
| Oxidation Inhibitor | 0.1 to 5 | 0.1 to 1.5 |
| Wear Inhibitor | 0.2 to 3 | 0.5 to 1 |
| Friction Modifier | 0.01 to 5 | 0.01 to 1.5 |
| Rust Inhibitor | 0.01 to 5 | 0.01 to 1.5 |
| Viscosity Modifier | 0.1 to 2 | 0.1 to 1 |
| (solid polymer basis) | | |
| Pour Point Depressant | 0 to 5 | 0.01 to 1.5 |
| Foam Inhibitor | 0.001 to 3 | 0.001 to 0.15 |

### EXAMPLES

The following illustrative examples are intended to be non-limiting.

### Example 1

### Distillation of Crude Propylene Oligomer

Oligomerization of propylene was carried out in a autoclave reactor for a time sufficient to generate 10 gallons of product. The hydrocarbon phase containing product and n-heptane, after washing and drying, was vacuum distilled to remove n-heptane and provide a stripped oligomer product. The stripped oligomer product was then vacuum distilled (about 1.5 torr) using a protruded packed distillation column (36″×2″) and 10 fractions of distilled product were recovered. Each fraction was analyzed was analyzed by GC and FIMS for carbon number distribution and ¹H NMR for isomerization level. The results are summarized in Table 2. The branching index can be defined as the % ratio of integral values of the methyl group (CH3) protons compared to the sum of the methylene (—CH2-), methinyl (—CH—) and methyl (—CH3) group protons.

TABLE 2

| | Nominal Carbon | Average Carbon | Branching |
|---|---|---|---|
| Distillation | Number Range | Number Range | Index |
| Fraction | (GC) | (FIMS) | (¹H NMR) |
| 1 | 15-18 | 18 | 59 |
| 2 | 18-21 | 20 | 59 |
| 3 | 21-24 | 21 | 59 |
| 4 | 24-27 | 24 | 58 |
| 5 | 27-30 | 27 | 58 |
| 6 | 30-33 | 30 | 57 |
| 7 | 33-36 | 33 | 57 |
| 8 | 36-39 | 35 | 56 |
| 9 | 39-45 | 40 | 58 |
| 10 | — | 48 | 58 |

### Example 2

### Preparation of Alkylphenols from Propylene Oligomers

Three different alkylphenols were separately prepared from combined distillation fractions of Example 1:
(1) Alkylphenol 1 from Fractions 1-2;
(2) Alkylphenol 2 from Fractions 4-6; and
(3) Alkylphenol 3 from Fractions 6-8.

The following general procedure was used to prepare each of the three alkylphenols. A 3-L three-neck round bottom flask equipped with a mechanical stirrer and a thermocouple was charged with propylene oligomer fractions:
(1) Alkylphenol 1: about 335 g each of Fractions 1 and 2 (about 2.5 moles total)
(2) Alkylphenol 2: about 250 g each of Fractions 4, 5 and 6 (about 2.0 moles total)
(3) Alkylphenol 3: about 250 g each of fractions 6, 7 and 8 (about 1.7 moles total)

Following the addition of the oligomer fractions, the stirrer was turned on and the flask charged with 1 kg of phenol. The reaction mixture was heated to 60° C. and then 200-250 g of AMBERLYST®-36 ion exchange resin (acid form, dried between 116° C. and 120° C. for 48-72 hours) was added. The flask was fitted with an air condenser and maintained under a nitrogen blanket. The reaction was monitored by GLPC and were deemed complete when no further decrease in propylene oligomer was observed (about 6 days for Alkylphenol 1, about 3 days for Alkylphenol 2, and about 4 days for Alkylphenol 3). The reaction mixture allowed to cool and subjected to vacuum filtration to remove the catalyst. Excess phenol was removed by vacuum distillation. The properties of the alkylphenols are summarized in Table 3.

TABLE 3

| | Alkylphenol 1 | Alkylphenol 2 | Alkylphenol 3 |
|---|---|---|---|
| Propylene Oligomer | 1-2 | 4-6 | 6-8 |
| Fractions | | | |
| Number Ave. Mol. | 372 | 463 | 532 |
| Wt.^{(a)}, Daltons | | | |
| % Phenol^{(b)} | 0 | 0 | 1.4 |
| % Lights^{(b)} | 0 | 0 | 1.1 |
| Ortho/Para Isomer Ratio^{(c)} | 33/67 | 75/25 | 45/55 |
| Flash Point^{(b)}, ° C. | 166 | 184 | 120 |
| Specific Gravity at 15° C. | 0.9009 | 0.8755 | 0.8812 |
| Kinematic Viscosity^{(e)} | 11.4 | 9.5 | 14.6 |
| at 100° C., mm²/s | | | |
| Hydroxyl Number, | 125 | 105 | 60 |
| mg KOH/g | | | |
| Equivalent Mol. Wt. | 449 | 534 | 935 |
| from Hydroxyl Number | | | |

^{(a)}Determined by electrospray ionization mass spectrometry
^{(b)}Determined by gas-liquid partition chromatography (GLPC)
^{(c)}Determined from IR peak heights
^{(d)}Determined with the Pensky-Martens Closed Cup Tester described in ASTM D93.
^{(e)}Determined according to ASTM D445

Distortionless enhancement by polarization transfer (DEPT) NMR was carried out to determine the total amount of CH₂ carbon atoms that are adjacent to the benzylic carbon atom which is attached to the hydroaromatic ring of Alkylphenols 1-3. A CH₂ carbon atom adjacent to the benzylic carbon atom which is attached to the hydroxyaromatic ring is calculated to appear at about 49 to 51 ppm in the ¹³C NMR spectrum. This chemical shift is unique among the CH₂ carbon atoms of propylene oligomer alkylphenols. This calculation was determined using CHEMDRAW® Ultra (Perkin Elmer). The results are summarized in Table 4.

TABLE 4

| | % of CH₂ Carbon Atoms Adjacent |
|---|---|
| | Benzylic Carbon Atom |
| Alkylphenol 1 | 0.3 |
| Alkylphenol 2 | 0.4 |
| Alkylphenol 3 | 0.7 |

The results show that greater than 99% of the CH₂ carbon atoms in Alkylphenols 1-3 are not adjacent to the benzylic carbon atom which is attached to the hydroxyaromatic ring.

### Example 3

### Synthesis of Alkylphenol Carboxylic Acid

A 4-L three-neck round bottom flask equipped with a Dean Stark trap was charged with Alkylphenol 2 of Example 8 (1411 g), xylene (706 g), a 45% aqueous KOH solution (365 g), and a foam inhibitor (0.2 g). The mixture was heated at 135° C. at reduced pressure (450 mm Hg) for 6 hours during which xylene and water were continuously distilled while xylene was returned to the mixture via the Dean Stark trap. The mixture was allowed to cool to ambient temperature under nitrogen. The mixture was then charged to a pressure vessel, heated to 140° C., and the reactor was pressurized with CO₂ (3 bars). After 4 hours, the reactor was depressurized and the reaction mixture was allowed to cool to ambient temperature.

The potassium carboxylate salt (1100 g) produced above was added to a round bottom flask followed by xylene (602 g) and the mixture was heated to 80° C. A 10% aqueous solution of H₂SO₄ (887 g) was added slowly to the mixture and the mixture was held at 70° C. for 30 minutes. The mixture was transferred to a separatory funnel and allowed to settle for 2 hours. After separation, the top layer containing the carboxylic acid in xylene was recovered.

The carboxylic acid had an acidity of 14.4 mg KOH/g, as measured by potentiometry, and a xylene content of 60.2 wt %.

### Example 4

### Preparation of an Overbased Carboxylate Detergent

A reactor was charged with slaked lime (60.3 g), methanol (72.3 g) and xylene (125 g). The carboxylic acid of Example 3 (2200 g) was added to the reactor and the temperature kept at 40° C. Then, a 50/50 mixture of acetic acid/formic acid (5.7 g) was added. After cooling to 30° C., CO₂ (12.8 g) was introduced in the reactor slowly while the temperature was ramped up from 30° C. to 40° C. The temperature was then raised to 128° C. during which methanol, water and some xylene is distilled off. Base oil (175.3 g) was added and then the resulting mixture was centrifuged to remove unreacted lime and other solids. The mixture was then heated at 170° C. under vacuum to remove xylene and afford an overbased carboxylate detergent.

The overbased carboxylate detergent had the following properties: 2.88% Ca, TBN=81 mg KOH/g, and a kinematic viscosity at 100° C. of 20.6 mm²/s.

### Example 5

### Preparation of an Overbased Phenate Detergent

A 4-L three-neck round bottom flask was charged with Alkylphenol 1 of Example 8 (881.6 g), 130 N base oil (357.9 g), an alkylaryl sulfonic acid (39.7 g), and a foam inhibitor (0.2 g). The mixture to warmed over 25 minutes to 110° C. and, while warming, hydrated lime (304 g) was added. Then, sulfur (902 g) was added and the reaction temperature increased to 150° C. over 20 minutes. After the sulfur addition, the pressure of the reactor was reduced to 680 mm Hg. Hydrogen sulfide gas that was produced during the sulfurization was trapped by two caustic soda bubblers. Then, ethylene glycol (46.6 g) was added over 45 minutes and the mixture was heated to 170° C. Over a 30 minute period, 2-ethylhexanol (393.6 g) was added which cooled the reaction to 162° C. The mixture was heated to 170° C. and additional ethylene glycol (76.4 g) was added over 1 hour. After ethylene glycol addition, the pressure was increased to 720 mm Hg and reaction conditions were maintained for 20 minutes. Maintaining the temperature at 170° C., the pressure was increased to 760 mm Hg. Then, CO₂ (9 g) was added over 30 minutes. Then, ethylene glycol (63.4 g) was added and the rate of CO₂ was increased to 0.8 g/min. The carbonation was stopped when about 96 g of CO₂ had been added. The solvent was then distilled at 215° C. and 30 mm Hg for 1 hour. The temperature was increased to 220° C. with a N₂ purge at 80 mm Hg over 1 hour. The product was vacuum filtered through CELITE® diatomaceous earth at 165° C. and the filtered overbased phenate was degassed under air over 4 hours at 5 L/h/kg of product at 150° C.

The overbased phenate detergent had the following properties: 10.5 wt % Ca; 3.15 wt % S; TBN=293 mg KOH/g; kinematic viscosity at 100° C. of 574 mm2/s.

### Example 6

### Preparation of an Olefin Sulfonate

Propylene oligomer distillation fraction 3 of Example 1 was sulfonated in a stainless steel, water jacketed, falling film tubular reactor (about 0.19″ ID×60″ length) using SO₃/air under the following conditions:

Propylene oligomer feed temperature=30° C.

Reactor temperature=40° C.

Air flow=200 L/h

Makeup air flow=11 L/h

SO₂ flow=16 L/h

SO₂ to SO₃ conversion=87%

Propylene oligomer feed rate=2.9 g/min

The resulting sulfonic acid had the following properties: 4.28 wt % H₂SO₄ and 35.18 wt % sulfonic acid (cyclohexylamine titration). The sulfonic acid was digested at 65° C. for 30 minutes to afford a digested sulfonic acid with the following properties: 3.99 wt % H₂SO₄ and 30.03 wt % sulfonic acid.

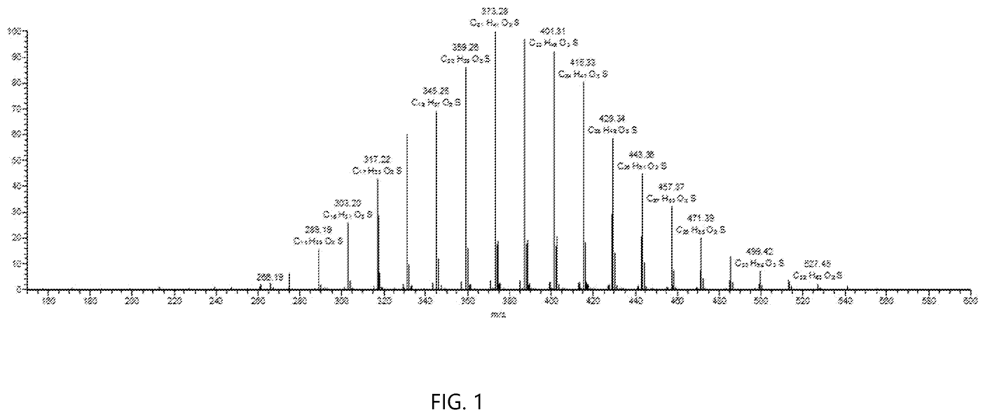

The digested sulfonic acid (222.3 g) was neutralized by addition of a 50 wt % aqueous NaOH solution (332 g) in portions between 25° C. and 51° C. over 30 minutes with stirring. The resulting sodium sulfonate was found to be 27.35 wt % active by Hyamine titration, pH=10.4 (about 1 wt. % in water solution). The ESI mass spectrum showed the major constituent in the sodium sulfonate composition to have a m/z charge ratio of 373 (see FIG. 1).

1. A method of treating a hydrocarbon-containing reservoir, comprising:
introducing into the reservoir a surfactant composition comprising:
an alpha olefin sulfonate or an internal olefin sulfonate, wherein the alpha olefin sulfonate or the internal olefin sulfonate is synthesized by i) oligomerizing a monomer comprising a C₃ to C₆ mono-olefin to form an oligomerization product and ii) sulfonating the oligomerization product.

2. The method of claim 1, wherein the i) oligomerizing step generates oligomers having at least 50 wt % C₁₂ to C₇₀ oligomers.

3. The method of claim 1, wherein the i) oligomerizing step generates oligomers at least 50 wt % C₁₆ to C₄₀ oligomers.

4. The method of claim 1, wherein the monomer comprises one or more of propylene and isobutylene.

5. The method of claim 1, wherein the surfactant further comprises:
a co-surfactant, a co-solvent, a base, an enhanced oil recovery flooding fluid, or a wellbore remediation fluid.

6. The method of claim 5, wherein the co-surfactant comprises of a secondary alkane sulfonate, internal olefin sulfonate, an alkoxylated alcohol sulfate, an alkoxylated alcohol carboxylate and glycerol sulfonate, a linear alkyl benzene sulfonate, heavy alkyl benzene sulfonate, sulfosuccinate, alkyl aromatic sulfonate, a nonionic alkoxylated alcohol, or alkyl aryl disulfonate, and mixtures thereof.

7. The method of claim 5, wherein the co-solvent is water, alcohol, or glycol.

8. The method of claim 5, wherein the base is a carbonate, a hydroxide, a bicarbonate, an ammonium, or an amine.

9. A surfactant for enhanced oil recovery, the surfactant comprising:
an alpha olefin sulfonate or an internal olefin sulfonate, wherein the alpha olefin sulfonate or the isomerized olefin sulfonate is synthesized by i) oligomerizing a monomer comprising a C₃ to Cᵣ mono-olefin to form an oligomerization product and ii) sulfonating the oligomerization product.

10. The surfactant of claim 9, wherein the i) oligomerizing step generates oligomers having at least 50 wt % C₁₂ to C₇₀ oligomers.

11. The surfactant of claim 9, wherein the i) oligomerizing step generates oligomers having at least 50 wt % C₁₆ to C₄₀ oligomers.

12. The surfactant of claim 9, wherein the monomer comprises one or more of propylene and isobutylene.

13. The surfactant of claim 9, further comprising:
co-surfactant, a co-solvent, a base, an enhanced oil recovery flooding fluid, or a wellbore remediation fluid

14. The surfactant of claim 13, wherein the co-surfactant comprises of a secondary alkane sulfonate, internal olefin sulfonate, an alkoxylated alcohol sulfate, an alkoxylated alcohol carboxylate and glycerol sulfonate, a linear alkyl benzene sulfonate, heavy alkyl benzene sulfonate, sulfosuccinate, alkyl aromatic sulfonate, a nonionic alkoxylated alcohol, or alkyl aryl disulfonate, and mixtures thereof.

15. The surfactant of claim 13, wherein the co-solvent is water, alcohol, or glycol.

16. The surfactant of claim 13, wherein the base is a carbonate, a hydroxide, a bicarbonate, an ammonium, or an amine.

17. A lubricating oil composition comprising:
a base oil; and
a succinimide dispersant synthesized by i) oligomerizing a monomer comprising a C₃ to C₆ mono-olefin to form a oligomerization product and ii) functionalizing the oligomerization product with an ethylenically saturated carboxylic acid group.

18. The lubricating oil composition of claim 17, wherein the ethylenically saturate carboxylic acid group is (meth)acrylic acid, methyl acrylate, maleic acid or anhydride, fumaric acid, or itaconic acid.

19. The lubricating oil composition of claim 17, wherein the ii) functionalizing step forms an alkyl-substituted succinic anhydride.

20. The lubricating oil composition of claim 17, wherein the monomer comprises one or more of propylene and isobutylene.

21. A dispersant composition comprising:
a succinimide dispersant synthesized by i) oligomerizing a monomer comprising a C₃ to C₆ mono-olefin to form a oligomerization product and ii) functionalizing the oligomerization product with an ethylenically saturated carboxylic acid group.

22. The dispersant of claim 21, wherein the ethylenically saturate carboxylic acid group is (meth)acrylic acid, methyl acrylate, maleic acid or anhydride, fumaric acid, or itaconic acid.

23. The dispersant of claim 21, wherein the ii) functionalizing forms an alkyl-substituted succinic anhydride.

24. The dispersant of claim 21, wherein the monomer comprises one or more of propylene and isobutylene.

25. A lubricating oil composition comprising:
a base oil; and
a detergent additive synthesized by i) oligomerizing a monomer comprising a C₃ to C₆ mono-olefin to form a oligomerization product and ii) alkylating a hydroxyaromatic compound with the oligomerization product.

26. The lubricating oil composition of claim 25, wherein the hydroxyaromatic compound is phenol, catechol, resorcinol, hydroquinone, pyrogallol, or cresol.

27. The lubricating oil composition of claim 25, wherein the detergent additive forms a salt with a metal.

28. The lubricating oil composition of claim 27, wherein the metal is Li, Na, K, Mg, Ca, Zn, Co, Mn, Zr, Ba, or B.

29. The lubricating oil composition of claim 25, wherein the monomer comprises one or more of propylene and isobutylene.

30. A detergent composition comprising:
a detergent additive synthesized by i) oligomerizing a monomer comprising a C₃ to C₆ mono-olefin to form a oligomerization product and ii) alkylating a hydroxyaromatic compound with the oligomerization product.

31. The detergent composition of claim 30, wherein the hydroxyaromatic compound is phenol, catechol, resorcinol, hydroquinone, pyrogallol, or cresol.

32. The detergent composition of claim 30, wherein the detergent additive forms a salt with a metal.

33. The detergent composition of claim 30, wherein the metal is Li, Na, K, Mg, Ca, Zn, Co, Mn, Zr, Ba, or B.

34. The detergent composition of claim 30, wherein the monomer comprises one or more of propylene and isobutylene.

35. A surfactant composition comprising:
an alcohol ether sulfate or an alcohol ether carboxylate synthesized by i) oligomerizing a monomer comprising a C₃ to C₆ mono-olefin to form a oligomerization product and ii) converting the oligomerization product to an alcohol.

36. The surfactant composition of claim 35, wherein the monomer comprises one or more of propylene and isobutylene.
